(19) United States
(12) Patent Application Publication
(10) Pub. No.: US 2018/0001113 A1
(43) Pub. Date: Jan. 4, 2018

(54) ULTRASOUND DEVICE WITH TOPICAL CONDUCTING MEDIUM

(71) Applicant: L'OREAL, Paris (FR)

(72) Inventor: John STREETER, Redmond, WA (US)

(73) Assignee: L'OREAL, Paris (FR)

(21) Appl. No.: 15/198,641

(22) Filed: Jun. 30, 2016

(51) Int. Cl. A61N 7/00; A61M 35/00; A61M 37/00

(52) U.S. Cl. CPC A61N 7/00; A61M 37/0092; A61M 35/00; A61N 2007/0034; A61M 2205/058

(57) ABSTRACT

A dermal device for providing ultrasound therapy to skin is provided including: a transducer assembly including housing structure having an inner surface defining a void and configured to contact an outer portion of an area of skin of a user without contacting an inner portion of the area of skin; and an ultrasound transducer supported by the housing and configured to be disposed proximate the void, wherein the ultrasound transducer is configured to provide ultrasound energy with a focal point towards the skin.

### BACKGROUND

### Field

The present application is directed to a system for providing focused dermal ultrasound therapy to select layers of a user's skin.

### SUMMARY

In an embodiment, a dermal device for providing ultrasound therapy to skin is provided including: a transducer assembly including housing structure having an inner surface defining a void and configured to contact an outer portion of an area of skin of a user without contacting an inner portion of the area of skin; and an ultrasound transducer supported by the housing and configured to be disposed proximate the void, wherein the ultrasound transducer is configured to provide ultrasound energy with a focal point towards the skin.

In an embodiment, the ultrasound transducer is configured to be adjusted within the device housing relative to the skin, wherein the focal point is modified based on the adjustment.

In an embodiment, the ultrasound transducer is adjusted by adjusting the housing, wherein the focal point is modified based on the adjustment of the housing.

In an embodiment, a dermal topical is configured fill the void and to serve as an ultrasound conductive medium and a therapeutic agent, wherein the dermal topical is one of a cosmetic, toner, cream, serum, or similar agent.

In an embodiment, the dermal topical has a viscosity similar to that of water.

A system for providing ultrasound therapy to skin is provided including: an ultrasound transducer configured to interface with one of at least two cartridges; and a cartridge having a cartridge shape such that the cartridge is configured to create an offset between the ultrasound transducer and an area of skin of a user; wherein the ultrasound transducer is configured to provide ultrasound energy with a focal point towards the area of skin, and wherein the focal point is based on the offset.

In an embodiment, the cartridge further includes a reservoir configured to hold a dermal topical.

In an embodiment, the dermal topical is configured to serve as a conductive medium within the cartridge.

In an embodiment, the cartridge further includes a semipermeable membrane configured to interface with the area of skin.

In an embodiment, the semipermeable membrane is configured to allow application of the dermal topical simultaneous with the ultrasound therapy to the area of skin.

In an embodiment, the semipermeable membrane is configured to partially interface with the area of skin, wherein the focal point is modified based on the semipermeable membrane.

In an embodiment, a cartridge for use in a system for providing ultrasound therapy to skin is provided including: a cartridge shape such that the cartridge is configured to create an offset between an ultrasound transducer and an area of skin of a user, wherein the ultrasound transducer is configured to provide ultrasound energy with a focal point towards the area of skin based on the offset.

In an embodiment, the cartridge further includes a reservoir configured to hold a dermal topical.

In an embodiment, the cartridge further includes a semipermeable membrane configured to interface with the area of skin, wherein the semipermeable membrane is configured to allow application of the dermal topical simultaneous with the ultrasound therapy to the area of skin.

In an embodiment, the cartridge further includes a first portion for conducting the ultrasound and a second portion for dispensing a dermal topical.

### BRIEF DESCRIPTION OF THE DRAWINGS

A more complete appreciation of the embodiments and many of the attendant advantages thereof will be readily obtained as the same becomes better understood by reference to the following detailed description when considered in connection with the accompanying drawings, wherein:

FIG. 1A is a drawing of a cross-section in an x-z plane of a dermal device having a housing and at least one ultrasound transducer having a shape that is configured to create a void when interfaced with skin according to an example;

FIG. 1B is a drawing of a cross-section in an x-z plane of a dermal device interfacing with the skin, and the dermal device further includes an adjuster configured to vertically adjust the ultrasound transducer(s) within the housing according to an example;

FIG. 1C is a drawing of a cross-section in an x-z plane of a dermal device interfacing with the skin, and the dermal device including one or more adjusters configured to vertically lengthen the housing according to an example;

FIG. 2A is a drawing in an x-z plane of a cross-section of a cartridge having an open surface configured to interface with the skin

FIG. 2B is a drawing in an x-z plane of a cross-section of a cartridge having a permeable membrane configured to interface with the skin according to an example;

FIG. 2C is a drawing in an x-z plane of a cross-section of a cartridge having a partial permeable membrane configured to interface with the skin according to an example;

FIG. 2D is a drawing in an x-z plane of a cross-section of a cartridge having an extension portion configured to interface with the skin according to an example;

FIG. 2E is a drawing in an x-z plane of a cross-section of a system for providing focused dermal ultrasound therapy including the dermal device and the cartridge of FIG. 2A according to an example;

FIG. 2F is a drawing of a cross-section of the system for providing focused dermal ultrasound therapy with the cartridge of FIG. 2B configured to create an offset between the dermal device and the skin when in use according to an example;

FIG. 2G is a drawing of a cross-section of the system for providing focused dermal ultrasound therapy with the cartridge of FIG. 2C configured to modify the focal point in the skin according to an example;

FIG. 2H is a drawing of a cross-section of the system for providing focused dermal ultrasound therapy with the cartridge of FIG. 2D according to an example;

FIG. 3A is a drawing of an underside of the dermal device as in FIG. 1A that is configured to interface with the skin according to an example;

FIG. 3B is a drawing of an underside of the system including the dermal device having the cartridge of FIG. 2B that is configured to interface with the skin according to an example;

FIG. 3C is a drawing of an underside of the system including the dermal device having the cartridge of FIG. 2C that is configured to interface with the skin according to an example;

FIG. 3D is a drawing in a x-y plane of an underside of the system having the cartridge of FIG. 2D that is configured to interface with the skin according to an example;

FIG. 4 is a drawing of a cross-section in an x-z plane of a set of layers of the skin according to an example;

FIG. 5A shows a flow chart describing a method for applying a topical and delivering ultrasound energy to a user's skin according to an example;

FIG. 5B shows a flow chart describing a method for delivering ultrasound energy to a user's skin based on a cartridge type according to an example; and

FIG. 5C shows a flow chart describing a method for delivering ultrasound energy to a user's skin based on a user input according to an example.

### DETAILED DESCRIPTION

Ultrasound energy can be used to condition and provide dermatological therapy to a user's skin. Focused ultrasound energy can be created in different scenarios. In a first scenario, an array of ultrasound transducers can be arranged in a curved shape to create a focal point of the ultrasound energy. In a second scenario, a material can be added in front of one or more ultrasound transducers to create the focal point of the ultrasound energy.

Two embodiments are presented for modifying the focal point of the ultrasound energy to target a specific layer of the skin (e.g. stratum corneum) within different layers of the user's skin. In a first embodiment, a dermal device for enhancing a dermal therapy regimen is disclosed that can be configured to simultaneously provide a dermal topical and transmit the ultrasound energy at an optimal focal point to a specific layer of a user's skin for the dermatological therapy. Using the dermal device with a dermal topical tailored to a specific layer of a user's skin, an amount of skin (e.g. stratum corneum) disruption is expected to enhance the dermatological therapy.

In a second embodiment, a system for providing focused dermal ultrasound therapy is disclosed which includes a cartridge that can be configured to simultaneously apply the dermal topical and modify the focal point of the ultrasound energy to target a specific layer of a user's skin for the dermatological therapy.

In an aspect the dermal topical provides an active agent and serves as a medium for ultrasound conduction. The dermal topical or topical can include a therapeutic or conditioning agent such as a toner (e.g. L'Oreal Paris Hydrafresh), a skin cleanser (e.g. Clarisonic Sonic Radiance), as well as any other applicable topical for conditioning skin. In an aspect the topical can be configured to have a viscosity similar to that of water in order to promote conduction of the ultrasound energy.

The dermal device can modify a focal point of the ultrasound energy to a user's skin in several ways. In an embodiment, the dermal device can modify a focal point by changing an offset of an ultrasound transducer to the skin. In an example, the dermal device can create the offset by extending a housing of the dermal device. In an example, the dermal device can create the offset by incorporating a cartridge that interfaces with the skin. In an aspect the cartridge can have a full or partial permeable membrane interfacing with the skin and be filled with the topical. In an aspect the cartridge can have a corresponding offset with respect to the topical and the dermal therapy regimen.

In an embodiment, the dermal device can modify a focal point by changing a medium of ultrasound conduction between an ultrasound transducer and the skin. In an aspect the cartridge can have a partial permeable membrane that only allows ultrasound conduction to a partial amount of skin.

Referring now to the drawings, wherein like reference numerals designate identical or corresponding parts throughout the several views.

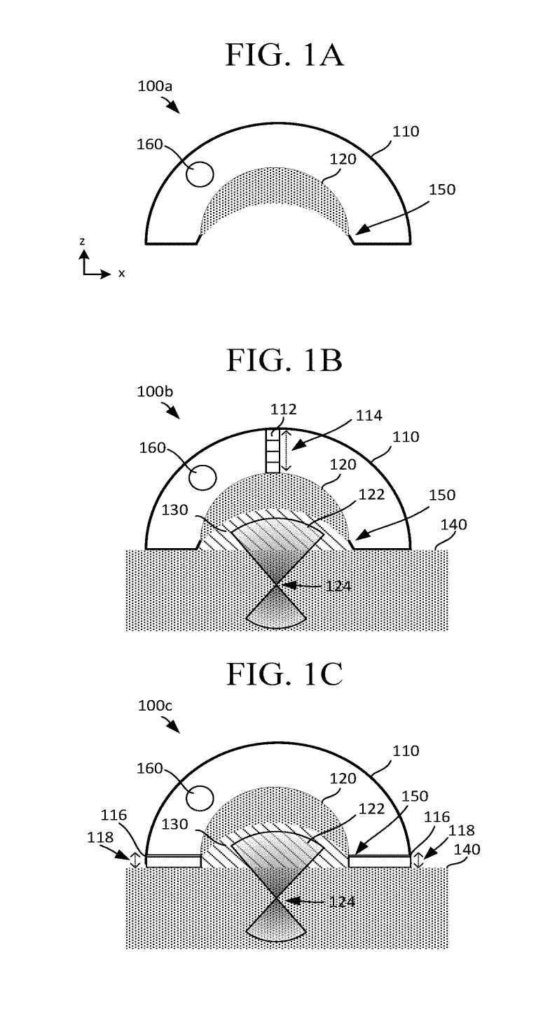

### FIGS. 1A-C

FIG. 1A is a drawing of a cross-section in an x-z plane of a dermal device 100a having a housing 110 and at least one ultrasound transducer 120 configured to create an offset 150 or void with the skin when the dermal device 100a is pressed against the skin according to an example. The housing 110 can include a user interface 160, a power source such as a battery or a connection an external power source such as an electrical outlet. In this example, the offset 150 is determined by a portion of the housing 110 configured to interface with the skin. The ultrasound transducer 120 can have an array of transducers configured to produce a focal ultrasound energy at a therapeutic frequency range for dermal applications such as a range of 30-300 KHz, consistent with Low Frequency Dermal Delivery, or 4-10 MHz, consistent with Dermal MicroFocused Ultrasound.

Each ultrasound transducer 120 can be configured to have a concave shape or the array of transducers can be configured to have a concave shape. Each ultrasound transducer 120 can be configured to create ultrasound energy 122 with a focal point 124 in a user's skin 140. The focal point 124 can be within one or more layers of a set of layers of the skin.

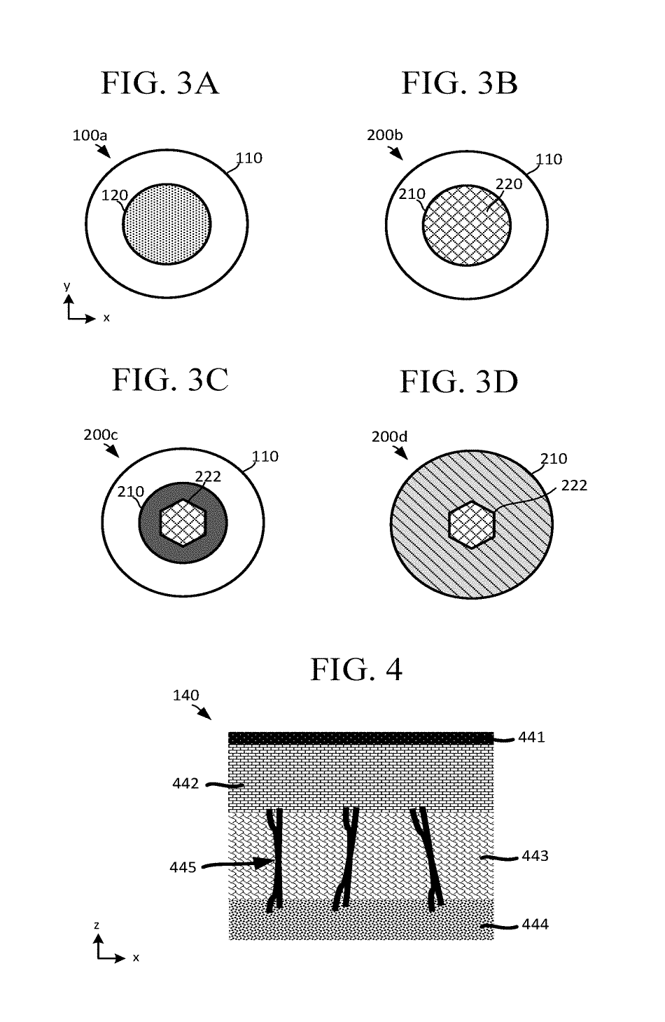

As shown in FIG. 4, the set of layers of the skin 140 includes an epidermis layer 441, a dermis layer 442, a fat cells layer 443, and a muscle layer 444 overlaid respectfully. The set of layers of the skin 140 further includes a set of septae 445 connecting the dermis layer 442 and the muscle layer 444. The set of layers of the skin may vary in thicknesses between individuals. Typical thickness of the epidermis layer 441 is between 0.05-1.2 mm, the dermis layer 442 is between 1-4 mm, and the fat cells layer 443 is between 2-9 mm.

In an example, the user interface 160 can have a display and a set of buttons (not shown) for a user input. The user input can include one or more control operations for using the dermal device including a start operation, a therapy duration, an intensity of the ultrasound energy, as well as other user functions such as setting a focal point of the ultrasound energy. The housing 110 can be configured to modify the focal point 124 of the ultrasound energy 122 in a different layer of the skin in several ways.

FIG. 1B shows a first example of a dermal device 100b including an adjuster 112 configured to vertically adjust 114 the ultrasound transducer 120 within the housing 110. A topical 130 can be manually applied to the skin 140 and can be configured to serve as both a conducting material for ultrasound energy 122 as well as the therapeutic or conditioning agent. The vertical adjustment 114 is configured to modify the offset 150 on a 1:1 ratio, but can be any another ratio for adjusting precision of the adjustment. Examples of the adjuster 112 include a mechanical screw that can be configured to be manually adjusted and an electro-mechanical stepper motor that can be configured to automatically adjust based on the dermal therapy regimen. The adjuster 112 can be aided by a contact sensor (not shown) in order to determine and modify the offset 150. The adjuster 112 can also be set by the user interface 160.

FIG. 1C shows a second example of a dermal device 100c including an adjuster 116 configured to vertically lengthen 118 the housing 110 according to an example. The vertical lengthening 118 of the adjuster 116 is configured to modify the offset 150 on a 1:1 ratio, but can be any another ratio for adjusting precision of the adjustment. Examples of the adjuster 116 include a mechanical screw configured to be manually adjusted and an electro-mechanical stepper motor that can be configured to automatically adjust based on the dermal therapy regimen. In an example, the adjuster 116 can extend and collapse similar to a telescope. In another example, the adjuster 116 can be a threaded ring around the housing 110, such that when the housing is rotated relative to the threaded ring, their displacement extends. The adjuster 116 can also be aided by a contact sensor (not shown) in order to determine and modify the offset 150. The adjuster 116 can also be set by the user interface 160.

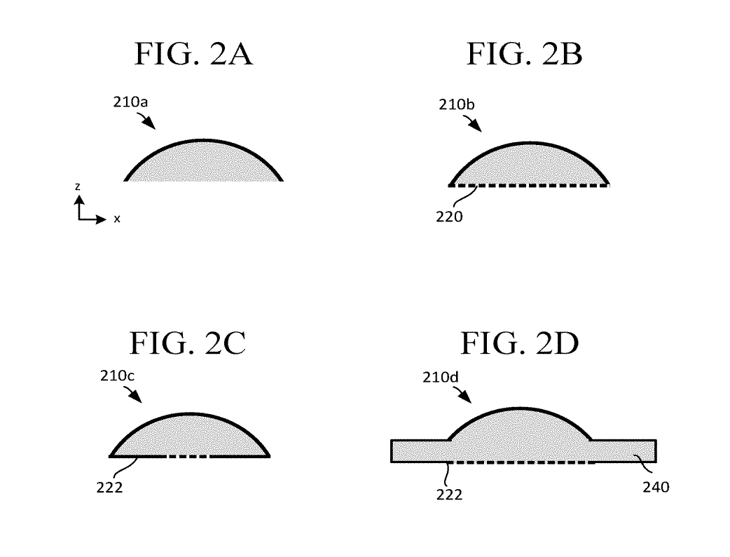

### FIGS. 2A-D

In a second embodiment, the dermal device can be part of a system 200 for providing focused dermal ultrasound therapy to select layers of a user's skin. The system 200 can include the dermal device 100 and a removable cartridge or cartridge 210 according to an example. The cartridge can be coupled to either the housing or the ultrasound transducer in several ways including by a friction fit, a bayonet fit, a tongue and grove fit, and a magnetic fit. In an aspect, coupling of the cartridge to the system can indicate a cartridge type. In an aspect, detection (e.g. by an electrical contact or reed switch) of proper coupling of the cartridge to the system can act as a safety measure and control operation of the dermal device. In another example, either the housing or the cartridge can include a sensor such as a contact sensor or a proximity sensor (not shown) configured to control activation of the ultrasound energy. In another example, the user interface 160 can be configured to control activation of the ultrasound energy.

Examples of the cartridge 210 include a cartridge 210a having an open surface configured to interface with the skin (see FIG. 2A), a cartridge 2100b having a permeable membrane 220 configured to interface with the skin (see FIG. 2B), a cartridge 210c having a partial permeable membrane 222 configured to interface with the skin (see FIG. 2C) and a cartridge 210d having an extension portion 240 configured to interface with the skin (see FIG. 2D).

The cartridge 210 can be loaded with the topical 130 such that the ultrasound energy 122 can be in communication with the skin when in use. In an aspect the permeable membrane 220 and partial permeable membrane 222 are configured to withhold the topical 130 and to allow for conduction of the ultrasound energy 122 to the skin.

In an aspect the permeable membrane 220 and partial permeable membrane 222 allows for pre-loading of the topical 130. In an aspect the partial permeable membrane 222 can be configured to modify the focal point by modifying the ultrasound conduction to the skin. For an example the partial permeable membrane 222 can be configured to only allow a portion 230 of the ultrasound energy 122 to pass to the skin. In an aspect, the partial permeable membrane 222 can be configured to modify a depth of a focal point 126, which can be in a different layer of the skin.

The cartridge can be configured to have a portion for conducting the ultrasound and a portion for dispensing the topical. As shown in FIG. 2D, the cartridge 210 can have an extension portion 240 that extends beyond the ultrasound transducer 120. The extension portion 240 can be configured to source the topical for application to the skin. For example, the extension portion 240 can be a fluid sac, a reservoir, or can be made of a material like a sponge. In an aspect, the extension portion 240 can be configured to create an offset 250 with the housing 110.

### FIGS. 2E-H

The cartridge 210 can be configured with the system 200 in several ways. In a first example, a cartridge 210a can be removable and can have a shape configured to be flush with the housing 110 (see FIG. 2E). In this example, the cartridge 210 is configured to fill the void with the skin and match the offset 150 with the skin 140 as determined by the housing 110. In a second example, the cartridge 210 can have a shape configured to create an offset 250 with the skin 140 as determined by the cartridge 210 and to modify the focal point 124 of the ultrasound energy (see FIGS. 2F, 2G and 2H). In an aspect, the cartridge 210 can have a shape configured to extend the offset 250 with the skin 140 such that the focal point of the ultrasound energy 122 varies in the one or more layers of the set of layers skin 140 (see FIGS. 2F, 2G and 2H).

In an aspect the focal point and/or the offset 250 can be based on a dermal therapy regimen. In one example, the focal point and/or the offset can be based on the topical 130 used for the dermal therapy regimen. In another example, the offset can be based on the ultrasound energy 122 used for the dermal therapy regimen.

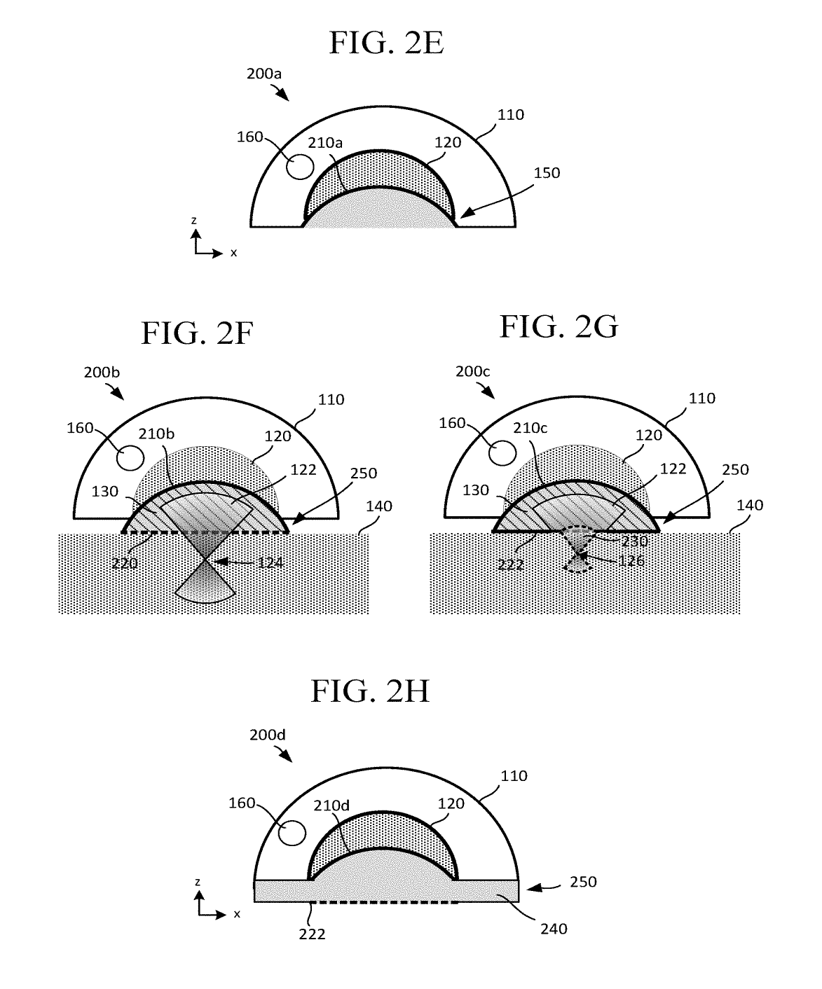

### FIGS. 3A-D

Examples of interfacing portions of the system 200 and the dermal device 100 are shown in FIGS. 3A-3D.

FIG. 3A is a drawing in a x-y plane of an underside of the dermal device 100a which is configured to interface with the skin according to an example. An underside of the ultrasound transducer 120 is shown creating a void with a circular or cylindrical shape; however, the void may have other shapes.

FIG. 3B is a drawing in a x-y plane of an underside of the system 200b that is configured to interface with the skin according to an example. An outline of the cartridge 210 is shown along with the permeable membrane 220 according to an example.

FIG. 3C is a drawing in a x-y plane of an underside of the system 200c having the cartridge 210c with the partial permeable membrane 222 which is configured to interface with the skin according to an example. In an aspect only a permeable portion of the partial permeable membrane 222 can be configured to allow for the ultrasound conduction to the skin.

FIG. 3D is a drawing in a x-y plane of an underside of the system 200d having the cartridge 210d with the extension portion 240 which is configured to interface with the skin according to an example.

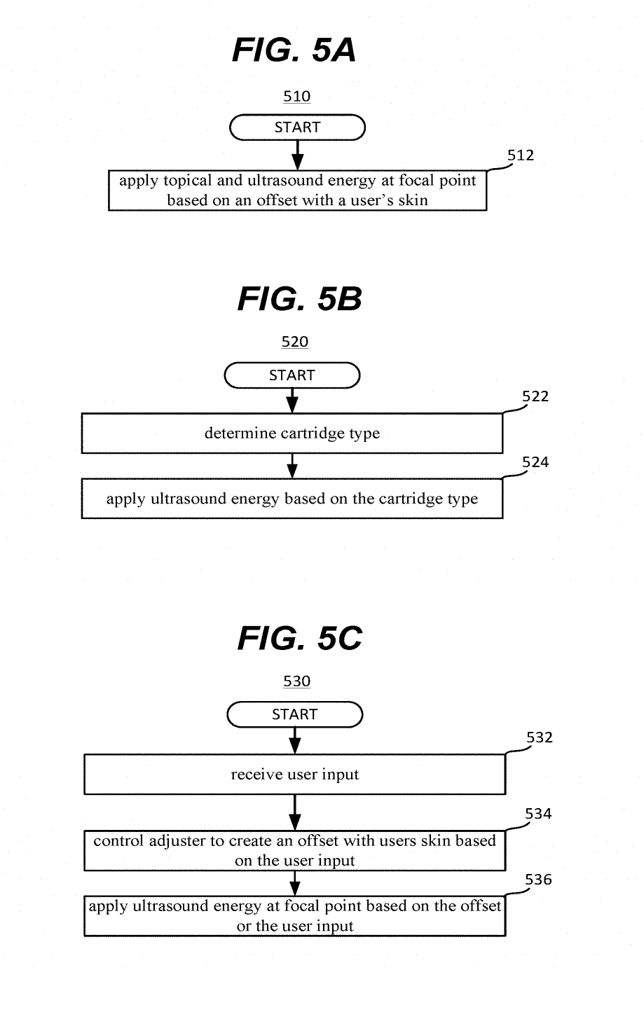

### FIGS. 5A-5C

A method can be used for delivering ultrasound energy according to several scenarios. FIG. 5A shows a flow chart describing a method 510 for applying a topical and delivering ultrasound energy to a user's skin according to an example. At step 512, a dermal device is configured to apply a topical and ultrasound energy at a focal point based on an offset with a user's skin.

FIG. 5B shows a flow chart describing a method 520 for delivering ultrasound energy to a user's skin based on a cartridge type according to an example. At step 522, a dermal device is configured to determine a cartridge type. At step 524, the dermal device is configured to apply a topical and ultrasound energy at a focal point based on the cartridge type.

FIG. 5C shows a flow chart describing a method 530 for delivering ultrasound energy to a user's skin based on a user input according to an example. At step 532, a dermal device is configured to receive a user input. At step 534, the dermal device is configured control an adjuster to create an offset with the user's skin based on the user input. At step 536, the dermal device is configured to apply ultrasound energy at a focal point based on the offset or the user input.

Obviously, numerous modifications and variations of the present invention are possible in light of the above teachings. It is therefore to be understood that within the scope of the appended claims, the invention may be practiced otherwise than as specifically described herein.

1. A dermal device for providing ultrasound therapy to skin, the dermal device comprising:
a transducer assembly including housing structure having an inner surface defining a void and configured to contact an outer portion of an area of skin of a user without contacting an inner portion of the area of skin; and
an ultrasound transducer supported by the housing and configured to be disposed proximate the void,
wherein the ultrasound transducer is configured to provide ultrasound energy with a focal point towards the skin.

2. The dermal device of claim 1, wherein the ultrasound transducer is configured to be adjusted within the device housing relative to the skin,
wherein the focal point is modified based on the adjustment.

3. The dermal device of claim 2, wherein the ultrasound transducer is adjusted by adjusting the housing,
wherein the focal point is modified based on the adjustment of the housing.

4. The dermal device of claim 1, wherein a dermal topical is configured fill the void and to serve as an ultrasound conductive medium and a therapeutic agent,
wherein the dermal topical is one of a cosmetic, toner, cream, serum, or similar agent.

5. The dermal device of claim 1, wherein the dermal topical has a viscosity similar to that of water.

6. A system for providing ultrasound therapy to skin, the system comprising:
an ultrasound transducer configured to interface with one of at least two cartridges; and
a cartridge having a cartridge shape such that the cartridge is configured to create an offset between the ultrasound transducer and an area of skin of a user;
wherein the ultrasound transducer is configured to provide ultrasound energy with a focal point towards the area of skin, and
wherein the focal point is based on the offset.

7. The system of claim 6, the cartridge further including a reservoir configured to hold a dermal topical.

8. The system of claim 7, wherein the dermal topical configured to serve as a conductive medium within the cartridge.

9. The system of claim 6, the cartridge further including a semipermeable membrane configured to interface with the area of skin.

10. The system of claim 9, wherein the semipermeable membrane is configured to allow application of the dermal topical simultaneous with the ultrasound therapy to the area of skin.

11. The system of claim 9, wherein the semipermeable membrane is configured to partially interface with the area of skin,
wherein the focal point is modified based on the semipermeable membrane.

12. A cartridge for use in a system for providing ultrasound therapy to skin, the cartridge comprising:
a cartridge shape such that the cartridge is configured to create an offset between an ultrasound transducer and an area of skin of a user,
wherein the ultrasound transducer is configured to provide ultrasound energy with a focal point towards the area of skin based on the offset.

13. The cartridge of claim 12, including a reservoir configured to hold a dermal topical.

14. The cartridge of claim 12, including a semipermeable membrane configured to interface with the area of skin,
wherein the semipermeable membrane is configured to allow application of the dermal topical simultaneous with the ultrasound therapy to the area of skin.

15. The cartridge of claim 12, including a first portion for conducting the ultrasound and a second portion for dispensing a dermal topical.
